(19) United States
(12) Patent Application Publication
(10) Pub. No.: US 2010/0323012 A1
(43) Pub. Date: Dec. 23, 2010

(54) Pharmaceutical Compositions Comprising NEP-Inhibitors, Inhibitors of the Endogenous Endothelin Producing System and Diuretics

(75) Inventors: Matthias STRAUB, JW Groet (NL); Klaus Witte, Hannover (DE); Dieter Ziegler, Hemmingen (DE); Yvan Fischer, Barsinghausen (DE)

Correspondence Address: CROWELL & MORING LLP;INTELLECTUAL PROPERTY GROUP, P.O. BOX 14300, WASHINGTON, DC, 20044-4300, US

(73) Assignee: Solvay Pharmaceuticals GmbH, Hannover (DE)

(21) Appl. No.: 12/872,049

(22) Filed: Aug. 31, 2010

Related U.S. Application Data
(63) Continuation of application No. 11/356,088, filed on Feb. 17, 2006.
(60) Provisional application No. 60/653,956, filed on Feb. 18, 2005.

(51) Int. Cl. A61K 31/55; A61K 9/28; A61P 9/00; A61P 1/16; A61P 13/12; A61P 9/04; A61P 9/10; A61P 9/12; B65D 83/04

(52) U.S. Cl. 424/474; 514/212.07; 206/528

(57) ABSTRACT

A novel combination therapy for cardiovascular diseases or conditions, including administering a synergistic combination of at least one inhibitor of neutral endopeptidase, at least one inhibitor of the endogenous endothelin producing system and at least one diuretic, preferably a thiazide diuretic or an adenosine A1 antagonist.

### CROSS REFERENCE TO RELATED APPLICATIONS

This application is a continuation of co-pending application Ser. No. 11/356,088, filed Feb. 17, 2006, now abandoned, which in turn claimed priority of U.S. provisional patent application No. 60/653,956, filed Feb. 18, 2005.

### BACKGROUND OF THE INVENTION

The present invention relates to a novel combination therapy for cardiovascular, renal and/or further diseases or conditions, in particular for cardiovascular diseases involving hypertension, by administering a synergistic combination of at least one inhibitor of neutral endopeptidase (=NEP), at least one inhibitor of the endogenous endothelin producing system and at least one diuretic. Thus, the invention also relates to novel pharmaceutical compositions comprising NEP inhibitors, inhibitors of the endogenous endothelin producing system, and diuretics and the use of said pharmaceutical composition in the prophylaxis or treatment of cardiovascular, renal and/or further diseases in mammals and humans.

The nature of cardiovascular, in particular hypertensive vascular, diseases is multifactorial. Combination therapy has been shown to address the multiple pathophysiologic factors that play a role in blood pressure elevation, including blood volume, vasoconstriction, and the impact of sympathetic nervous system and Renin-Angiotensin-Aldosterone-System (=RAAS) activity (see e.g. M. R. Weir, American Journal of Hypertension 11 (1998) 163S-169S), potentially resulting in both greater reduction in blood pressure and in lowered risks for target-organ damage. The use of a fixed, low-dose combination agent could also offer lower doses of each component than those that may be necessary with monotherapy, thus reducing the risks of dose-dependent adverse events and associated compliance problems.

U.S. Pat. No. 4,749,688 (=EP 254,032) discloses that NEP inhibitors can lower blood pressure under conditions where angiotensin converting enzyme (=ACE) inhibitors as a monotherapy are relatively ineffective.

In congestive heart failure, as a result of the decreased cardiac output and the increase in peripheral resistance, back-pressure phenomena of the blood occur in the pulmonary circulation and the heart itself. As a result, an increased wall tension of the heart muscle occurs in the area of the auricles and chambers. In such a situation, the heart functions as an endocrine organ and secretes, inter alia, the atrial natriuretic peptide (=ANP) into the bloodstream. Due to its marked vasodilatory and natriuretic/diuretic activity, ANP brings about both a reduction in the peripheral resistance and a decrease in the circulating blood volume. The consequence is a marked pre- and afterload decrease. This constitutes an endogenous cardioprotective mechanism. This positive endogenous mechanism is limited in that ANP has only a very short half-life in the plasma. The reason for this is that the hormone is very rapidly broken down by NEP. Therefore, pharmacological NEP inhibition rises ANP levels and thus promotes this cardioprotective mechanism. Due to a disease-related reduced output of the heart in congestive heart failure, a reflex increase in peripheral vascular resistance occurs. As a result, the heart muscle must begin to pump against an increased afterload. In a vicious cycle, this results in increased strain on the heart and worsens the situation further. The increase in the peripheral resistance is mediated, inter alia, by the vasoactive peptide endothelin. Endothelin (=ET) is the strongest presently known endogenous vasoconstrictory substance and is formed from the precursor big endothelin (=bigET) with participation of the endothelin converting enzyme (=ECE). Therefore, pharmacological inhibition lowers the levels of vasoconstrictive ET.

For these reasons, a combination of compounds having NEP-inhibiting activity with compounds capable of inhibiting the endogenous endothelin producing system or compounds with dual inhibiting activities on NEP and the endogenous endothelin producing system would seem to provide added value in the therapy of cardiovascular diseases like essential hypertension, pulmonary hypertension and/or congestive heart failure. As a result of inhibition of the endogenous endothelin producing system, formation of endothelin would be prevented and thus an increase in peripheral resistance would be counteracted, to result in a relief of the strain on the heart muscle. Inhibition of the ANP degrading enzyme NEP can thus lead to higher ANP levels and an increased duration of action of ANP. This will lead to a reinforcement of the ANP-mediated endogenous cardioprotective mechanism of action. However, because NEP may also be involved in ET degradation, a pure NEP inhibition would, in addition to the desired increase in the ANP levels, also lead to an unfavorable increase in the ET levels. For this reason, a mixed profile with dually acting inhibition of NEP and of the endogenous endothelin producing system is to be regarded as particularly favorable, since it prevents both the breakdown of the natriuretically/diuretically acting ANP (by NEP-blockade), and simultaneously inhibits the formation of ET. As a result, the adverse attendant effect of pure NEP-inhibitors (increase in the endothelin levels) no longer comes to bear.

Compounds with a dually acting combined inhibitory effect on NEP and the endogenous endothelin producing system, i.e. benzazepine-, benzoxazepine- and benzothiazepine-N-acetic acid derivatives, are known from U.S. Pat. No. 5,677,297 (=EP 733,642). Further favorable pharmacological properties of compounds falling within the structural scope of U.S. Pat. No. 5,677,297 are known from documents U.S. Pat. No. 5,783,573 (=EP 830,863), U.S. Pat. No. 6,482,820 (=WO 00/48601) and US 2003/0040512 (=WO 01/03699).

Phosphonic acid substituted benzazepinone-N-acidic acid derivatives with a combined inhibitory effect on NEP and the endogenous endothelin producing system are disclosed in U.S. Pat. No. 5,952,327 (=EP 916,679).

Amidomethyl-substituted 1-(carboxyalkyl)-cyclopentylcarbonylamino-benzazepine-N-acetic acid derivatives which are useful e.g. for the prophylaxis and/or treatment of cardiovascular conditions or diseases, are disclosed in published US patent application no. 2005/0119247 (=WO 2005/030795).

Published US patent application no. 2004/0162345 (=WO 02/094176) discloses that certain compounds, including those disclosed in U.S. Pat. No. 5,677,297 and U.S. Pat. No. 5,952,327 may inhibit the endogenous endothelin producing system via an inhibition of metalloprotease IGS5. The metalloprotease IGS5 is also known as human soluble endopeptidase (=hSEP) and is described e.g. in US 2004/0162345. Further, US 2004/0162345 discloses the use of compounds with combined NEP/hSEP inhibitory activity for the prophylaxis or treatment of inter alia cardiovascular diseases.

Diuretics are drugs which act on the kidney to promote excretion of water and electrolytes, particularly sodium. These drugs are e.g. widely used in treating edematous conditions such as those associated with cardiovascular diseases. Certain combinations of cardiovascular active agents with diuretics are already known.

Published US patent application no. 2004/0192584 (=WO 2004/082636) discloses a combination of an aldosterone receptor antagonist and a neutral endopeptidase inhibitor.

Published US patent application no. 2004/0186083 provides a combination of an aldosterone receptor antagonist and an endothelin receptor antagonist and/or endothelin converting enzyme inhibitor.

Published US patent application no. 2004/0266698 discloses pyran derivatives as both ACE- and NEP inhibitors.

International patent application WO 2006/000564 refers to pharmaceutical compositions comprising NEP-inhibitors, inhibitors of the endogenous endothelin producing system and AT₁ receptor antagonists.

### SUMMARY OF THE INVENTION

It is the object of the present invention to provide a novel combination therapy for cardiovascular diseases, renal diseases and/or further diseases with enhanced efficacy and a favorable safety profile.

It has now surprisingly been found that a combination of at least one NEP-inhibitor, at least one inhibitor of the endogenous endothelin producing system and additionally at least one diuretic, provides still further enhanced efficacy and a favorable safety profile in the prophylaxis or treatment of cardiovascular diseases, renal diseases and/or further diseases.

The invention therefore relates in a first aspect to pharmaceutical compositions comprising pharmacologically effective quantities of each of
a) at least one NEP-inhibitor as a first active agent,
b) at least one inhibitor of the endogenous endothelin producing system as a second active agent and
c) at least one diuretic as a third active agent.

The pharmaceutical compositions according to the invention may further and preferably comprise conventional pharmaceutically acceptable auxiliaries and/or carriers.

Inhibitors of the endogenous endothelin producing system can be selected from the group consisting of inhibitors of ECE, inhibitors of hSEP and dually acting compounds capable of inhibiting ECE and hSEP.

In the pharmaceutical compositions according to the invention, the subcombination of at least one NEP-inhibitor a) and at least one inhibitor of the endogenous endothelin producing system b) can preferably be realized by a dually acting compound of general Formula I,

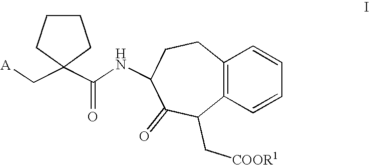

wherein
R¹ is hydrogen or a group forming a biolabile carboxylic acid ester
A represents a group selected from the subgroups (a),

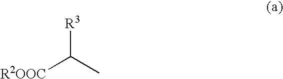

wherein
R² is hydrogen or a a group forming a biolabile carboxylic acid ester and
R³ is a phenyl-C₁₋₄-alkyl group which can optionally be substituted in the phenyl ring by C₁₋₄-alkyl, C₁₋₄-alkoxy or halogen; or a naphthyl-C₁₋₄-alkyl group; or
(b),

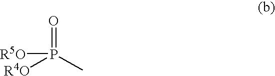

wherein
R⁴ is hydrogen or a group forming a biolabile phosphonic acid ester and
R⁵ is hydrogen or a group forming a biolabile phosphonic acid ester; or (c)

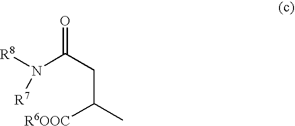

wherein
R⁶ is hydrogen or a group forming a biolabile carboxylic acid ester,
R⁷ is hydrogen, C₁₋₄-alkyl or C₁₋₄-hydroxyalkyl, the hydroxyl group of which is optionally esterified with C₂₋₄-alkanoyl or an amino acid residue, and
R⁸ is C₁₋₄-alkyl; C₁₋₄-alkoxy-C₁₋₄-alkyl; C₁₋₄-hydroxyalkyl, which is optionally substituted by a second hydroxyl group and the hydroxyl groups of which are each optionally esterified with C₂₋₄-alkanoyl or an amino acid residue; (C₀₋₄-alkyl)₂-amino-C₁₋₆-alkyl; C₃₋₇-cycloalkyl; C₃₋₇-cycloalkyl-C₁₋₄-alkyl; phenyl-C₁₋₄-alkyl, the phenyl group of which is optionally substituted 1-2 times by C₁₋₄-alkyl, C₁₋₄-alkoxy and/or halogen; naphthyl-C₁₋₄-alkyl; C₃₋₆-oxoalkyl; phenylcarbonylmethyl, the phenyl group of which is optionally substituted 1-2 times by C₁₋₄-alkyl, C₁₋₄-alkoxy and/or halogen, or 2-oxoazepanyl, or
R⁷ and R⁸ together are C₄₋₇-alkylene, the methylene groups of which are optionally replaced 1-2 times by carbonyl, nitrogen, oxygen and/or sulfur and which are optionally substituted once by hydroxy, which is optionally esterified with C₂₋₄-alkanoyl or an amino acid residue; C₁₋₄-alkyl; C₁₋₄-hydroxyalkyl, the hydroxyl group of which is optionally esterified with C₂₋₄-alkanoyl or an amino acid residue; phenyl or benzyl,
   and/or physiologically compatible salts of acids of Formula I and/or physiologically compatible acid addition salts of compounds of Formula Ic.

Where the substituents in the compounds of Formula I are or contain C₁₋₄-alkyl groups, these may be straight-chain or branched. Where biolabile ester forming groups in the compounds of Formula I are or contain lower alkyl groups, these may be straight-chain or branched and contain usually 1 to 4 carbon atoms. Where the substituents contain halogen, fluorine, chlorine or bromine, fluorine or chlorine are particularly suitable. Where substituents contain C₂₋₄-alkanoyl, this may be straight-chain or branched. Acetyl is preferred as C₂₋₄-alkanoyl.

Where substituents are biolabile ester forming groups, these as a rule represent prodrugs of the active drug principle. Prodrugs are therapeutic agents which are inactive per se but are transformed into one or more active metabolites. Prodrugs are bioreversible derivatives of drug molecules used to overcome some barriers to the utility of the parent drug molecule. These barriers include, but are not limited to, solubility, permeability, stability, pre-systemic metabolism and targeting limitations (see e.g. Medicinal Chemistry Principles and Practice, 1994, ISBN 0-85186-494-5, Ed.: F. D. King, p. 215; J. Stella, “Prodrugs as therapeutics”, Expert Opin. Ther. Patents, 14(3), 277-280, 2004; P. Ettmayer et al., “Lessons learned from marketed and investigational prodrugs”, J. Med. Chem., 47, 2393-2404, 2004).

Suitable physiologically compatible salts of free acids or partial esters of Formula I include their alkali metal, alkaline earth metal or ammonium salts, for example sodium or calcium salts or salts with physiologically compatible, pharmacologically neutral organic amines such as, for example, diethylamine or tert.-butylamine.

Preferred are the compounds of general Formula Ia,

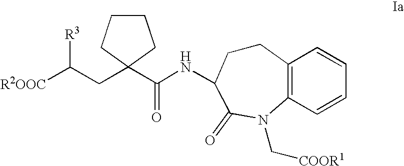

wherein R¹, R² and R³ have the above meanings, and physiologically compatible salts of acids of Formula Ia. Compounds of Formula Ia are known, e.g. from U.S. Pat. No. 5,677,297, the disclosure of which is incorporated herein by reference in its entirety. Preferred salts of compounds of Formula Ia are e.g. disclosed in published US patent application no. 2005/0038012 (=WO 03/059939) which is incorporated herein by reference. The compounds of Formula Ia contain two asymmetric or chiral carbon atoms, namely the carbon atom which is in the 3 position of the ring framework (=3-position) and bears the amide side-chain, and the carbon atom of the amide side-chain which bears the radical R³ (=2′-position). The compounds can therefore exist in several optically active stereoisomeric forms or as a racemate. According to the present invention the mixtures of stereoisomers or the racemic mixtures and the isomerically pure compounds of Formula Ia may be used.

The compounds of Formula Ia are optionally esterified dicarboxylic acid derivatives. Depending on the form of administration, biolabile monoesters, particularly compounds in which R² is a group forming a biolabile ester and R¹ is hydrogen, or dicarboxylic acids are preferred, the latter being particularly suitable for i.v. administration. Groups which can be cleaved under physiological conditions in vivo, releasing bioavailable derivatives of the compounds of Formula Ia, are suitable as groups forming biolabile carboxylic acid esters R¹ and R². Suitable examples of this are C₁₋₄-alkyl groups, in particular methyl, ethyl, n-propyl and isopropyl; C₁₋₄-alkyloxy-C₁₋₄-alkyloxy-C₁₋₄-alkyl groups, in particular methoxyethoxymethyl; C₃₋₇-cycloalkyl groups, in particular cyclohexyl; C₃₋₇-cycloalkyl-C₁₋₄-alkyl groups, in particular cyclopropylmethyl; N,N-di-(C₀₋₄-alkyl)amino-C₁₋₆-alkyl groups; phenyl or phenyl-C₁₋₄-alkyl groups optionally substituted in the phenyl ring once or twice by halogen, C₁₋₄-alkyl or C₁₋₄-alkoxy or by a C₁₋₄-alkylene chain bonded to two adjacent carbon atoms; dioxolanylmethyl groups optionally substituted in the dioxolane ring by C₁₋₄-alkyl; C₂₋₆-alkanoyloxy-C₁₋₄-alkyl groups optionally substituted at the oxy-C₁₋₄-alkyl group by C₁₋₄-alkyl; double esters like 1-[[(C₁₋₄-alkyl)carbonyl]oxy]C₁₋₄-alkyl esters, e.g. (RS)-1-[[(isopropyl)carbonyl]oxy]ethyl or (RS)-1-[[(ethyl)carbonyl]oxy]-2-methylpropyl (for preparation see e.g. F. W. Sum et al., Bioorg. Med. Chem. Lett. 9 (1999) 1921-1926 or Y. Yoshimura et al., The Journal of Antibiotics 39/9 (1986) 1329-1342); carbonate esters like 1-[[(C₄₋₇-cycloalkyloxy)carbonyl]oxy]C₁₋₄-alkyl esters, preferably (RS)-1-[[(cyclohexyloxy)carbo-nyl]oxy]ethyl (=cilexetil; for preparation see e.g. K. Kubo et al., J. Med. Chem. 36 (1993) 2343-2349, cited as “Kubo et al.” hereinafter)) or 2-oxo-1,3-dioxolan-4-yl-C₁₋₄-alkyl esters which optionally contain a double bond in the dioxolan ring, preferably 5-methyl-2-oxo-1,3-dioxolen-4-yl-methyl (=medoxomil, for preparation see e.g. Kubo et al.) or 2-oxo-1,3-dioxolan-4-yl-methyl (=(methyl)ethylenecarbonate). Where the group forming a biolabile ester represents an optionally substituted phenyl-C₁₋₄-alkyl group, this may contain an alkylene chain with 1 to 3, preferably 1, carbon atoms and preferably stands for optionally substituted benzyl, in particular for 2-chlorobenzyl or 4-chlorobenzyl. Where the group forming a biolabile ester represents an optionally substituted phenyl group, the phenyl ring of which is substituted by a lower alkylene chain, this may contain 3 to 4, preferably 3, carbon atoms and in particular be indanyl. Where the group forming a biolabile ester represents an optionally substituted C₂₋₆-alkanoyloxy-C₁₋₄-alkyl group, the C₂₋₆-alkanoyl group may be straight-chain or branched.

R¹ preferably has the meanings hydrogen, C₁₋₄-alkyl, p-methoxybenzyl, N,N-di-(C₀₋₄-alkyl)amino-C₁₋₆-alkyl, (RS)-1-[[(isopropyl)carbonyl]oxy]ethyl, (RS)-1-[[(ethyl)carbonyl]-oxy]-2-methylpropyl, (RS)-1-[[(cyclohexyloxy)carbonyl]oxy]ethyl, 5-methyl-2-oxo-1,3-dioxolen-4-yl-methyl, 2-oxo-1,3-dioxolan-4-yl-methyl or (RS)-1-[[(ethoxy)carbonyl]oxy]-ethyl.

R² preferably has the meanings hydrogen, ethyl, methoxyethoxymethyl, (RS)-1-[[(isopropyl)carbonyl]oxy]ethyl, (RS)-1-[[(ethyl)carbonyl]oxy]-2-methylpropyl, (RS)-1-[[(cyclohexyloxy)carbonyl]oxy]ethyl, 5-methyl-2-oxo-1,3-dioxolen-4-yl-methyl, 2-oxo-1,3-dioxolan-4-yl-methyl or (RS)-1-[[(ethoxy)carbonyl]oxy]ethyl.

More preferred are the compounds of Formula Ia which are selected from the group consisting of 2-[1-(1-carboxymethyl-2-oxo-2,3,4,5-tetrahydro-1H-benzo[b]azepin-3-ylcarbamoyI)-cyclopentylmethyl]-4-phenyl-butyric acid ethyl ester [alternative name: 3-[1-{2′-(ethoxycarbonyl)}-4′-phenylbutyl]-cyclopentan-1-carbonylamino}-2,3,4,5-tetrahydro-2-oxo-1H-1-benzazepin-1-acetic acid] of Formula II,

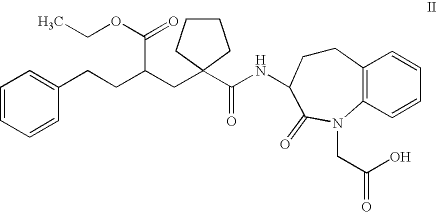

2-[1-(1-carboxymethyl-2-oxo-2,3,4,5-tetrahydro-1H-benzo[b]azepin-3-ylcarbamoyl)-cyclopentylmethyl]-4-naphthalen-1-yl-butyric acid ethyl ester [alternative name: 3-[1-{2-(ethoxycarbonyl)-4-(1-naphthyl)butyl]cyclopentyl}carbonyl)amino]-2-oxo-2,3,4,5-tetrahy-dro-1H-1-benzazepin-1-yl}acetic acid] of Formula III,

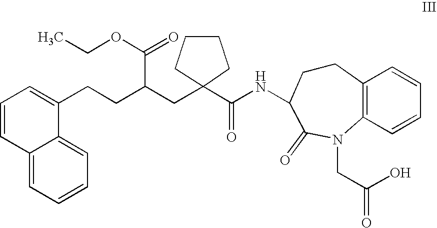

2-[1-(1-carboxymethyl-2-oxo-2,3,4,5-tetrahydro-1H-benzo[b]azepin-3-ylcarbamoyl)-cyclopentylmethyl]-4-phenyl-butyric acid of Formula IV,

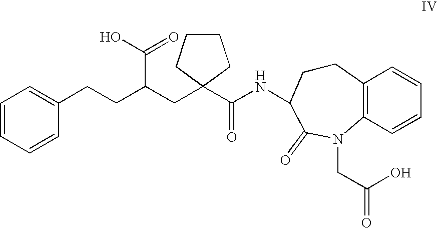

2-[1-(1-carboxymethyl-2-oxo-2,3,4,5-tetrahydro-1H-benzo[b]azepin-3-ylcarbamoyl)-cyclopentylmethyl]-4-naphthalen-1-yl-butyric acid of Formula V,

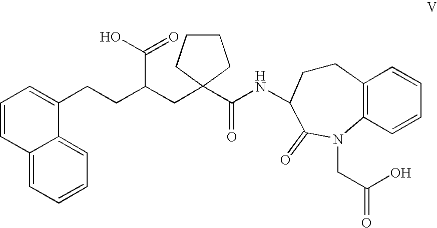

and physiologically compatible salts of the acids of Formulas II, III, IV and/or V. The compounds of Formulas II, III, IV and V are especially suited in their 3S,2′R forms. Most preferred is the compound of Formula II in its 3S,2′R form, also known as “daglutril” or “SLV306”. The compounds of Formula Ia are known, for example, from U.S. Pat. No. 5,677,297 (=EP 733,642) which is incorporated herein by reference in its entirety, and can be produced according to the production processes disclosed or referenced in this document or analogously to said production processes.

Further, compounds of general Formula Ib,

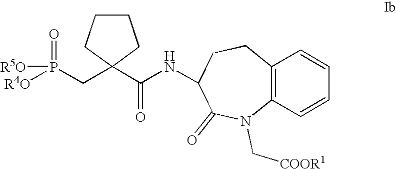

wherein R¹, R⁴ and R⁵ have the meanings given above, or physiologically compatible salts of acids of Formula Ib can be used as dually acting compounds capable of inhibiting NEP and the endogenous endothelin producing system. The compounds of Formula Ib are known, for example, from U.S. Pat. No. 5,952,327 (=EP 916 679), the disclosure of which is incorporated herein by reference in its entirety, and can be produced according to the production processes disclosed or referenced in this document or analogously to said production processes.

Suitable groups R¹ forming biolabile carboxylic acid esters in compounds of Formula Ib are the same as those specified for compounds of Formula Ia above.

Groups R⁴ and R⁵ suitable as groups forming biolabile phosphonic acid esters are those which can be removed under physiological conditions in vivo with release of the respective phosphonic acid function. For example, groups which are suitable for this purpose are lower alkyl groups, C₂-C₆-alkanoyloxymethyl groups optionally substituted on the oxymethyl group by lower alkyl, or phenyl or phenyl-lower alkyl groups whose phenyl ring is optionally mono- or polysubstituted by lower alkyl, lower alkoxy or by a lower alkylene chain bonded to two adjacent carbon atoms. If the group R⁴ and/or R⁵ forming a biolabile ester is or contains lower alkyl, this can be branched or unbranched and can contain 1 to 4 carbon atoms. If R⁴ and/or R⁵ are an optionally substituted alkanoyloxymethyl group, it can contain a preferably branched alkanoyloxy group having 2 to 6, preferably 3 to 5, carbon atoms and can, for example, be a pivaloyloxy-methyl radical (=tert-butylcarbonyloxymethyl radical). If R⁴ and/or R⁵ are an optionally substituted phenyl-lower alkyl group, this can contain an alkylene chain having 1 to 3, preferably 1, carbon atoms. If the phenyl ring is substituted by a lower alkylene chain, this can contain 3 to 4, in particular 3, carbon atoms and the substituted phenyl ring is in particular indanyl.

The compounds of the formula Ib contain an asymmetric or chiral carbon atom, namely the carbon atom carrying the amide side chain in the 3-position of the benzazepine structure. The compounds can thus be present in two optically active stereoisomeric forms or as a racemate. The present invention includes both the racemic mixtures and the isomerically pure compounds of the formula I. If R⁴ and R⁵ in compounds of the formula Ib are not hydrogen and in each case have different meanings, the phosphorus atom of the phosphonic acid group can also be chiral. The invention also relates to the isomer mixtures and isomerically pure compounds of the formula Ib formed as a result of chiral phosphorus atoms.

When compounds of Formula Ib are used according to the invention, (3-{[1-(benzyloxy-ethoxy-phosphorylmethyl)-cyclopentanecarbonyl]-amino}-2-oxo-2,3,4,5-tetrahydro-benzo[b]azepin-1-yl)-acetic acid tert-butyl ester and isobutyric acid 1-[[1-(−1-carboxymethyl-2-oxo-2,3,4,5-tetrahydro-1H-benzo[b]azepin-3-ylcarbamoyl)-cyclopentyl-methyl]-(1-isobutyryloxy-ethoxy)-phosphinoyloxy]-ethyl ester are preferred. Both of said compounds are particularly preferred when the stereochemistry at the chiral carbon atom (see above) is “S”, namely in their “(3S)” configuration. The compounds of Formula Ib are known, for example, from U.S. Pat. No. 5,952,327 (=EP 916 679) and can be produced according to the production processes disclosed or referenced in this document or analogously to said production processes.

Also preferred are the compounds of general Formula Ic,

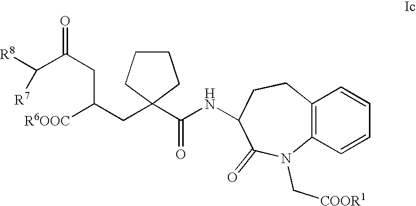

wherein R¹, R⁶, R⁷ and R⁸ have the above meanings, and physiologically compatible salts of acids of Formula Ic and/or physiologically compatible acid addition salts of compounds of Formula Ic, for the use as dually acting compounds capable of inhibiting NEP and the endogenous endothelin producing system in pharmacological compositions according to the invention. The compounds of Formula Ic are known, for example, from published US patent application no. 2005/0119247 (=WO 2005/030795), which is incorporated herein by reference in its entirety, and can be produced according to the production processes disclosed or referenced in this document or analogously to said production processes.

Where in compounds of Formula Ic the substituents R⁷ and/or R⁸ contain basic groups, in particular nitrogen, the compounds of Formula Ic may also occur in the form of acid addition salts. Physiologically compatible acid addition salts of compounds of Formula Ic are their conventional salts with inorganic acids, for example sulfuric acid, phosphoric acid or hydrohalic acids, preferably hydrochloric acid, or with organic acids, for example lower aliphatic monocarboxylic, dicarboxylic or tricarboxylic acids such as maleic acid, fumaric acid, tartaric acid, citric acid, or with sulfonic acids, for example lower alkanesulfonic acids such as methanesulfonic acid.

Suitable groups R¹ forming biolabile carboxylic acid esters in compounds of Formula Ic are the same as those specified for compounds of Formula Ia above. Suitable groups R⁶ forming biolabile carboxylic acid esters in compounds of Formula Ic are the same as specified for groups R² in compounds of Formula Ia above.

R⁷ preferably has the meanings hydrogen, methyl, ethyl, 2-hydroxyethyl or 3-hydroxypropyl, each hydroxyl group optionally being esterified with C₂₋₄-alkanoyl or an amino acid residue.

Where R⁸ has the meaning (C₀₋₄-alkyl)₂-amino-C₁₋₆-alkyl, one or two C₀₋₄-alkyl groups can independently of each other be present. More specifically, “(C₀₋₄-alkyl)₂-amino-C₁₋₆-alkyl” expressly comprises the meanings “(C₀)₂-alkylamino-C₁₋₆-alkyl”, “(C₀)(C₁₋₄)-alkylamino-C₁₋₆-alkyl” and “(C₁₋₄)₂-alkylamino-C₁₋₆-alkyl”. “(C₀)₂-alkylamino-C₁-6-alkyl” is meant to denominate an unsubstituted primary (═—NH₂) amino group bonded to C₁₋₆-alkyl(en); “(C₀)(C₁₋₄)-alkylamino-C₁₋₆-alkyl” is meant to denominate a secondary amino group monosubstituted by (C₁₋₄-alkyl and bonded to C₁₋₆-alkyl(en); “(C₁₋₄₂-alkylamino-C₁₋₆-alkyl” is meant to denominate a tertiary amino group disubstituted by (C₁₋₄)-alkyl and bonded to C₁₋₆-alkyl(en). R⁸ preferably has the meanings isopropyl; methoxyethyl; 2-hydroxyethyl or 3-hydroxypropyl, each hydroxyl group optionally being esterified with C₂₋₄-alkanoyl or an amino acid residue; 3-acetyloxy-n-propyl; cyclopropylmethyl; 2-methoxybenzyl, 4-methoxybenzyl; 4-methoxyphenylethyl; 2,4-dimethoxybenzyl; 1-naphthylmethyl; 3-oxo-1,1-dimethylbutyl; phenyl-2-oxoethyl; 2-(4-methoxyphenyl)-2-oxoethyl; 3-(2-oxoazepanyl); (C₀₋₄-alkyl)₂-amino-C₁₋₆-alkyl, in particular dimethylamino-n-propyl, (methyl)aminoethyl, amino-n-propyl, amino-n-butyl or amino-n-pentyl.

Where R⁷ and R⁸ together are C₄₋₇-alkylene, the methylene groups of which are optionally replaced or optionally substituted, in each case morpholine; piperidine; 4-ketopiperidine; 4-hydroxypiperidine, optionally being esterified with C₂₋₄-alkanoyl or an amino acid residue at the hydroxyl group; piperazine or pyrrolidine is preferred.

Where in the compounds of Formula Ic hydroxyl groups are esterified with amino acid residues, these amino acid residues may be derived from natural or non-natural, α- or β-amino acids. Suitable amino acids which can be used are for example selected from the group cosisting of alanine, 2-aminohexanoic acid (=norleucine), 2-aminopentanoic acid (=norvaline), arginine, asparagine, aspartic acid, cysteine, 3,4-dihydroxyphenylalanine (=dopa), glutamine, glutamic acid, glycine, histidine, isoleucine, leucine, lysine, methionine, ornithine (=2,5-diaminovaleric acid), 5-oxo-2-pyrrolidine-carbonic acid (=pyroglutamic acid), phenylalanine, proline, serine, threonine, thyronine, tryptophan, tyrosine and valine. Preferred are amino acid residues which are derived from alanine, asparagine, glutamine, glycine, isoleucine, leucine, lysine, ornithine, phenylalanine, proline and valine.

The compounds of Formula Ic contain two asymmetric or chiral carbon atoms, namely the carbon atom bearing the amide side chain in position 3 of the benzazepine skeleton (═C_{b}*) and the carbon atom bearing the radical “—COOR⁶” (═Cₐ*). The compounds can thus be present in a total of four stereoisomeric forms. The present invention comprises both the mixtures of stereoisomers and enantiomers, and also the isomerically pure compounds of Formula Ic. Isomerically pure compounds of Formula Ic are preferred. Particularly preferred are compounds of Formula Ic wherein the carbon atom bearing the amide side chain in position 3 of the benzazepine skeleton is in the “S” configuration. With respect to the chiral carbon atom “*Cₐ” bearing the radical “—COOR⁶”, the configuration of the compounds of Formula I which is preferred according to the invention in the context of this invention is provisionally assigned the configuration designation “rel1”. It can be derived by analogous observations of suitable compounds of known configuration that the preferred configuration “rel1” at the chiral center “*Cₐ” is probably likewise the “S” configuration.

Particularly preferred compounds of Formula Ic are selected from the group consisting of:
2-[1-(1-Carboxymethyl-2-oxo-2,3,4,5-tetrahydro-1H-benzo[b]azepin-3-ylcarbamoyl)-cyclopentylmethyl]-N-(3-dimethylamino-propyl)-N-methyl-succinamic acid;
2-{[1-({[1-(carboxymethyl)-2-oxo-2,3,4,5-tetrahydro-1H-1-benzazepin-3-yl]amino}carbo-nyl)cyclopentylynethyl}-4-[isopropyl(methypamino]-4-oxobutanoic acid;
2-{[1-({[1-(carboxymethyl)-2-oxo-2,3,4,5-tetrahydro-1H-1-benzazepin-3-yl]amino}carbo-nyl)cyclopentyl]methyl}-4-(dimethylamino)-4-oxobutanoic acid;
2-{[1-({[1-(carboxymethyl)-2-oxo-2,3,4,5-tetrahydro-1H-1-benzazepin-3-yl]amino}carbo-nyl)cyclopentyl]methyl}-4-(diethylamino)-4-oxobutanoic acid;
2-{[1-({[1-(carboxymethyl)-2-oxo-2,3,4,5-tetrahydro-1H-1-benzazepin-3-yl]amino}car-bonyl)cyclopentyl]methyl}-4-[(2-hydroxyethyl)(methypamino]-4-oxobutanoic acid;
2-{[1-Q[1-(carboxymethyl)-2-oxo-2,3,4,5-tetrahydro-1H-1-benzazepin-3-yl]amino}car-bonyl)cyclopentyl]methyl}-4-[(3-hydroxypropyl)(methypamino]-4-oxobutanoic acid;
2-{[1-({[1-(carboxymethyl)-2-oxo-2,3,4,5-tetrahydro-1H-1-benzazepin-3-yl]amino}-carbonyl)cyclopentyl]methyl}-4-(4-hydroxypiperidin-1-yl)-4-oxobutanoic acid;
2-{[1-({[1-(carboxymethyl)-2-oxo-2,3,4,5-tetrahydro-1H-1-benzazepin-3-yl]amino}-carbonyl)cyclopentyl]methyl}-4-oxo-4-[4-(L-valyloxy)piperidin-1-yl]butanoic acid;
2-{[1-({[1-(carboxymethyl)-2-oxo-2,3,4,5-tetrahydro-1H-1-benzazepin-3-yl]amino}-carbonyl)cyclopentyl]methyl}-4-morpholin-4-yl-4-oxobutanoic acid;
2-{[1-({[1-(carboxymethyl)-2-oxo-2,3,4,5-tetrahydro-1H-1-benzazepin-3-yl]amino}-carbonyl)cyclopentyl]methyl}-4-oxo-4-(4-oxopiperidin-1-yl)butanoic acid;
4-[bis(2-hydroxyethyl)amino]-2-{0-({[1-(carboxymethyl)-2-oxo-2,3,4,5-tetrahydro-1H-1-benzazepin-3-yl]amino}carbonyl)cyclopentylynethyl}-4-oxobutanoic acid;
2-{[1-({[1-(carboxymethyl)-2-oxo-2,3,4,5-tetrahydro-1H-1-benzazepin-3-yl]amino}car-bonyl)cyclopentyl]methyl}-4-{ethyl[3-(ethylamino)propyl]amino}-4-oxobutanoic acid;
2-{[1-({[1-(carboxymethyl)-2-oxo-2,3,4,5-tetrahydro-1H-1-benzazepin-3-yl]amino]-carbonyl)cyclopentyl]methyl}-4-[[2-(dimethylamino)ethyl](methypamino]-4-oxobutanoic acid;
4-[(3-aminopropyl)(ethyl)amino]-2-{[1-({[1-(carboxymethyl)-2-oxo-2,3,4,5-tetrahydro-1H-1-benzazepin-3-yl]amino}carbonyl)cyclopentyl]methyl}-4-oxobutanoic acid,
2-{[1-({[1-(carboxymethyl)-2-oxo-2,3,4,5-tetrahydro-1H-1-benzazepin-3-yl]amino}-carbonyl)cyclopentyl]methyl}-4-{methyl[2-(methylamino)ethyl]amino}-4-oxobutanoic acid,
4-[(4-aminobutyl)(methyl)amino]-2-{[1-({[1-(carboxymethyl)-2-oxo-2,3,4,5-tetrahydro-1H-1-benzazepin-3-yl]amino}carbonyl)cyclopentyl]methyl}-4-oxobutanoic acid;
4-[(4-aminobutyl)(ethyl)amino]-2-{[1-({[1-(carboxymethyl)-2-oxo-2,3,4,5-tetrahydro-1H-1-benzazepin-3-yl]amino}carbonyl)cyclopentyl]methyl}-4-oxobutanoic acid;
2-{[1-({[1-(carboxymethyl)-2-oxo-2,3,4,5-tetrahydro-1H-1-benzazepin-3-yl]amino}-carbonyl)cyclopentyl]methyl}-4-{methyl[3-(methylamino)propyl]amino}-4-oxobutanoic acid and
4-[(5-aminopentyl)(methyl)amino]-2-{[1-({[1-(carboxymethyl)-2-oxo-2,3,4,5-tetrahydro-1H-1-benzazepin-3-yl]amino}carbonyl)cyclopentyl]methyl}-4-oxobutanoic acid,
   together with their biolabile esters and physiologically compatible salts of acids of these compounds of Formula Ic and/or physiologically compatible acid addition salts of these compounds of Formula Ic. 2-[1-(1-Carboxymethyl-2-oxo-2,3,4,5-tetrahydro-1H-benzo-[b]azepin-3-ylcarbamoyl)-cyclopentylmethyl]-N-(3-dimethylamino-propyl)-N-methyl-succinamic acid or its biolabile esters or physiologically compatible salts of its acids are particularly preferred compounds of Formula Ic.

Diuretics which can be used according to the present invention are understood to comprise any physiologically compatible salt, solvate, prodrug or ester thereof and may be selected from adenosine A1 antagonists, thiazide diuretics, thiazide analogues, loop diuretics, potassium sparing diuretics, carbonic anhydrase inhibitors and/or ethacrynic acid. Combinations of diuretics can also be used as a third active agent c). Thiazide diuretics or adenosine A1 antagonists are the preferred diuretics according to the invention.

Suitable adenosine A1 antagonists can be selected from the group consisting of 1,3-dipropyl-8-cyclopentylxanthine (DPCPX); 4-[(2-phenyl-7H-pyrrolo[2,3-d]pyrimidin-4-yl)amino]-trans-cyclohexanol; (4S)-4-hydroxy-1-(2-phenyl-7H-pyrrolo[2,3-d]pyrimidin-4-yl)-L-prolinamide; 8-cyclopentyl-3-N-[3-((3-(4-fluorosulfonyl)benzoyl)-oxy)-propyl]-1-N-propyl-xanthine (FSCPX); BG-9928 (CAS No. 340021-17-2); CPX (CAS No. 102146-07-6); FK-352 (CAS No. 143881-08-7); FK-453 (CAS No. 121524-18-3); FK-838 (CAS No. 131185-37-0); FR-166124 (CAS No. 171050-45-6); KW-3902 (CAS No. 136199-02-5); N-0861 ([+/−]N6-endo-norbornan-2-yl-9-methyladenine, CAS No. 141696-90-4); WRC-0342 (CAS No. 175097-37-7); WRC-0571 (8-(N-methylisopropyl)amino-N-6-(5′-endohydroxy-endonorbornyl)-9-methyladenine, CAS No. 175097-35-5); naxifylline (CAS Nos. 166374-48-7 and 166374-49-8) or any physiologically compatible tautomers, salts, solvates, prodrugs or esters thereof. 4-[(2-phenyl-7H-pyrrolo[2,3-d]pyrimidin-4-yl)amino]-trans-cyclohexanol may be administered as its methanesulfonate salt (=SLV320, CAS No. 685561-51-7). (4S)-4-hydroxy-1-(2-phenyl-7H-pyrrolo[2,3-d]-pyrimidin-4-yl)-L-prolinamide may be administered as its methanesulfonate salt. 4-[(2-phenyl-7H-pyrrolo[2,3-d]pyrimidin-4-yl)amino]-trans-cyclohexanol and (4S)-4-hydroxy-1-(2-phenyl-7H-pyrrolo[2,3-d]pyrimidin-4-yl)-L-prolinamide or their physiologically compatible salts are the preferred adenosine A1 antagonists according to the invention. 4-[(2-phenyl-7H-pyrrolo[2,3-d]pyrimidin-4-yl)amino]-trans-cyclohexanol or its physiologically compatible salts are e.g. known from U.S. Pat. Nos. 6,686,336; 6,800,633 and 6,878,716 (=WO 99/62518). (4S)-4-hydroxy-1-(2-phenyl-7H-pyrrolo[2,3-d]pyrimidin-4-yl)-L-prolinamide or its physiologically compatible salts are e.g. known from U.S. Pat. Nos. 6,664,252 and 6680,332 and published US patent application no. 2002/094574 (=WO 02/057267).

Suitable thiazide diuretics can be selected from the group consisting of althiazide, bemetizide, bendroflumethiazide, benzylhydrochlorothiazide, benzthiazide, buthiazide, chlorothiazide, cyclothiazide, cyclopenthiazide, ethiazide, hydrochlorothiazide, hydroflumethiazide, methylclothiazide, paraflutizide, polythiazide, teclothiazide, trichlormethiazide or any physiologically compatible tautomers, salts, solvates, prodrugs or esters thereof. Hydrochlorothiazide is the preferred thiazide diuretic.

Suitable thiazide analogue diuretics can be selected from the group consisting of chloraminofenamide, chlortalidone, clofenamide, clopamide, clorexolone, fenquizone, indapamide, mefruside, metolazone, quinethazone, tripamide, xipamide or any physiologically compatible tautomers, salts, solvates, prodrugs or esters thereof.

Suitable loop diuretics can be selected from the group consisting of azosemide, bumetanide, furosemide, piretanide, torsemide or any physiologically compatible tautomers, salts, solvates, prodrugs or esters thereof.

Suitable potassium sparing diuretics can be selected from the group consisting of amiloride, potassium canrenoate, spironolactone, triamterene or any physiologically compatible tautomers, salts, solvates, prodrugs or esters thereof.

Suitable carbonic anhydrase inhibitor diuretics can be selected from the group consisting of acetazolamide, brinzolamide, dichlorophenamide, dorzolamide, ethoxzolamide, indisulam, methazolamide, zonisamide or any physiologically compatible tautomers, salts, solvates, prodrugs or esters thereof.

The combination of at least one NEP-inhibitor, at least one inhibitor of the endogenous endothelin producing system and additionally at least one diuretic, has been found to provide still further enhanced efficacy and a favorable safety profile in the prophylaxis or treatment of cardiovascular diseases, renal diseases and/or further diseases.

As used herein the term “cardiocvascular diseases” comprises, e.g., acute coronary syndrome; acute heart failure; angina pectoris; angina abdominalis; arrhythmias; cardiac hypertrophy; cerebral infarction; cerebral ischemias; chronic heart failure; congestive heart failure; coronary heart disease; critical leg ischemia; hypertension, in particular essential hypertension, pulmonary hypertension, renal hypertension and/or hypertension associated with obesity, insulin resistance and/or diabetes; myocardial infarction; restenosis and/or stroke. The use of the combinations according to the invention in treating hypertension in its different forms and from different origins is preferred.

Renal diseases according to the invention may be of different origins, and may e.g. comprise renal diseases due to heart failure, diabetes or toxic agents. More specifically, renal diseases may comprise e.g. acute renal failure; chronic renal failure (chronic kidney disease), in particular diabetic nephropathy; and/or ischemic renal failure. Renal diseases due to toxic substances may e.g. be due to the prior administration of nephrotoxic radiocontrast media or cyclosporin. Cardiovascular diseases and renal diseases may occur jointly. It has been known in the art that renal diseases due to the administration of radiocontrast media may also be treated by the administration of adenosine A1 antagonists alone, including those adenosine A1 antagonists as described in the present invention. Further diseases may e.g. comprise liver fibrosis and/or liver cirrhosis.

The pharmaceutical compositions according to the invention can be prepared in a known manner and thus can be obtained as formulations suitable for enteral, such as oral or rectal, or parenteral administration to mammals or humans, comprising a therapeutically effective amount of the pharmacologically active agents, alone or in combination with one or more pharmaceutically acceptable auxiliaries and/or carriers, especially suitable for enteral or parenteral application. Pharmaceutical compositions for enteral or parenteral administration include, for example, in unit dosage forms, such as coated tablets, tablets, capsules or suppositories and also ampoules. These are prepared in a known manner, for example using conventional mixing, granulation, coating, solubilizing or lyophilizing processes. Typical oral formulations include coated tablets, tablets, capsules, syrups, elixirs and suspensions. Capsules may contain the active agents e.g. in form of powders, granules, pellets, beadlets or microtablets. For example, a pharmaceutical composition according to the invention may consist of from about 0.1% to 90%, preferably of from about 1% to about 80%, of the active agents, the rest being made up by pharmaceutically acceptable auxiliaries and/or carriers. Thus, pharmaceutical compositions for oral use can be obtained by combining the active compounds with solid excipients, if desired granulating the resulting mixture, and if required or necessary, processing the mixture or granulate into tablets or coated tablet cores after having added suitable auxiliary substances. Typical injectable formulations include solutions and suspensions.

In one embodiment of the pharmaceutical compositions according to the invention, the active agents (a), (b) and (c) can be obtained and administered together, e.g. in one combined unit dosage form like in one tablet or capsule, i.e. in a physical combination. In such a combined unit dosage form, the different active agents (a), (b) and (c) can be segregated from each other, e.g. by means of different layers in said tablet, e.g. by the use of inert intermediate layers known in the art; or by means of different compartments in said capsule. When a dually acting compound capable of inhibiting NEP and the endogenous endothelin producing system is used to embody the combination of active agents (a) and (b), the active agents [(a)+(b)] and (c) in the pharmaceutical composition can favorably be present in two separate dosage forms, usually complementary or balanced for combined use, e.g. as two different tablets or capsules, usually further comprising pharmaceutically acceptable auxiliaries and/or carriers, or in different compartments of one single capsule. Thus, in this embodiment at least the diuretic is present in a unit single dosage form physically segregated from the other active agent(s). The corresponding active agents or their pharmaceutically acceptable salts may also be used in form of their hydrates or include other solvents used for crystallization. A unit dosage form may be a fixed combination. A unit dosage form, in particular a fixed combination of the active agents (a), (b) and (c) is a preferred alternative of this embodiment.

In another embodiment the active agents (a), (b) and (c) can be obtained and administered in two or more separate unit dosage forms, e.g. in two or more tablets or capsules, the tablets or capsules being physically segregated from each other. The two or more separate unit dosage forms can be administered simultaneously or stepwise (separately), e.g. sequentially one after the other in either order. Thus, the active agents can be administered in either order at the same time or at different times spread over the day, the optimal dosage regimen usually being determined by prescription of a physician.

The typical pharmaceutically acceptable auxiliaries and/or carriers for use in the formulations described above are exemplified by: sugars such as lactose, sucrose, mannitol and sorbitol; starches such as cornstarch, tapioca starch and potato starch; cellulose and derivatives such as sodium carboxymethyl cellulose, ethyl cellulose and methyl cellulose; calcium phosphates such as dicalcium phosphate and tricalcium phosphate; sodium sulfate; calcium sulfate; polyvinylpyrrolidone; polyvinyl alcohol; stearic acid; alkaline earth metal stearates such as magnesium stearate and calcium stearate; stearic acid; vegetable oils such as peanut oil, cottonseed oil, sesame oil, olive oil and corn oil; non-ionic, cationic and anionic surfactants; ethylene glycol polymers; betacyclodextrin; fatty alcohols; and hydrolyzed cereal solids, as well as other non-toxic compatible fillers, binders, disintegrating agents, and other agents, e.g. talcum; buffers, preservatives, antioxidants, lubricants, flavoring and the like commonly used in pharmaceutical formulations.

In a specific embodiment of said first aspect, the invention also relates to a kit comprising in separate containers in a single package pharmaceutical dosage forms for use in combination, comprising,
i1) in one separate container a pharmaceutical dosage form comprising at least one neutral endopeptidase inhibitor and in a second separate container a pharmaceutical dosage form comprising at least one inhibitor of the endogenous endothelin producing system, or
i2) in one separate container a pharmaceutical dosage form comprising a dually acting compound capable of inhibiting neutral endopeptidase and the endogenous endothelin producing system, and
ii) in another separate container a pharmaceutical dosage form comprising at least one diuretic.

The kit form is particularly advantageous but not limited to the case when the separate components must be administered in different dosage forms or are administered at different dosage intervals. The dosage forms may favorably be oral formulations like tablets or capsules. The separate containers may e.g. be blister packs (in particular where the oral formulations are tablets or coated tablets), boxes or other containers commonly used to package pharmaceutical dosage forms. Preferred are alternatives of the kit which comprise in one separate container a pharmaceutical dosage form comprising a dually acting compound capable of inhibiting neutral endopeptidase and the endogenous endothelin producing system, in particular daglutril or 2-[1-(1-carboxymethyl-2-oxo-2,3,4,5-tetrahydro-1H-benzo[b]azepin-3-ylcarbamoyl)-cyclopentylmethyl]-N-(3-dimethylamino-propyl)-N-methyl-succinamic acid or any of their physiologically compatible salts or esters; and in another separate container a pharmaceutical dosage form comprising at least one diuretic, in particular hydrochlorothiazide, 4-[(2-phenyl-7H-pyrrolo[2,3-d]pyrimidin-4-yl)amino]-trans-cyclohexanol, or (4S)-4-hydroxy-1-(2-phenyl-7H-pyrrolo[2,3-d]pyrimidin-4-yl)-L-prolinamide or any of their physiologically compatible salts.

In a second aspect, the invention also relates to a use of at least one NEP-inhibitor in combination with at least one inhibitor of the endogenous endothelin producing system and at least one diuretic, for the preparation of a pharmaceutical composition or medicament for the prophylaxis or treatment of cardiovascular diseases, renal diseases and/or further diseases as discussed in more detail above.

In a third aspect, the invention relates to a method of treating or inhibiting a cardiovascular disease, a renal disease and/or a further disease, in mammals and humans, comprising administering to a subject in need thereof an effective amount of a combination of at least one NEP-inhibitor, at least one inhibitor of the endogenous endothelin producing system and at least one diuretic. Subjects in need of such treatment are in particular those humans or mammals who are suffering from or are susceptible to cardiovascular diseases, renal diseases and/or further diseases as discussed in more detail above.

In one specific embodiment of said third aspect, a fixed combination of a dually acting compound capable of inhibiting neutral endopeptidase and the endogenous endothelin producing system, and a diuretic can be used. Preferred alternatives of this specific embodiment are fixed combinations comprising daglutril and hydrochlorothiazide; daglutril and 4-[(2-phenyl-7H-pyrrolo[2,3-d]pyrimidin-4-yl)amino]-trans-cyclohexanol; daglutril and (4S)-4-hydroxy-1-(2-phenyl-7H-pyrrolo[2,3-d]pyrimidin-4-yl)-L-prolinamide; 2-[1-(1-carboxymethyl-2-oxo-2,3,4,5-tetrahydro-1H-benzo[b]azepin-3-ylcarbamoyl)-cyclopentylmethyl]-N-(3-dimethylamino-propyl)-N-methyl-succinamic acid and hydrochlorothiazide; 2-[1-(1-carboxymethyl-2-oxo-2,3,4,5-tetrahydro-1H-benzo[b]azepin-3-ylcarbamoyl)-cyclopentylmethyl]-N-(3-dimethylamino-propyl)-N-methyl-succinamic acid and 4-[(2-phenyl-7H-pyrrolo[2,3-d]pyrimidin-4-yl)amino]-trans-cyclohexanol; and 2-[1-(1-carboxymethyl-2-oxo-2,3,4,5-tetrahydro-1H-benzo[b]azepin-3-ylcarbamoyl)-cyclopentylmethyl]-N-(3-dimethylamino-propyl)-N-methyl-succinamic and (4S)-4-hydroxy-1-(2-phenyl-7H-pyrrolo[2,3-d]pyrimidin-4-yl)-L-prolinamide. Daglutril and 2-[1-(1-carboxymethyl-2-oxo-2,3,4,5-tetrahydro-1H-benzo[b]azepin-3-ylcarbamoyl)-cyclopentylmethyl]-N-(3-dimethylamino-propyl)-N-methyl-succinamic acid may each be present in the form of their their physiologically compatible salts or esters. Hydrochlorothiazide, 4-[(2-phenyl-7H-pyrrolo[2,3-d]pyrimidin-4-yl)amino]-trans-cyclohexanol and (4S)-4-hydroxy-1-(2-phenyl-7H-pyrrolo[2,3-d]pyrimidin-4-yl)-L-prolinamide may each be present in the form of their physiologically compatible salts.

### Description of the Test Method

The beneficial effects of the combination therapy according to the invention can be shown in suitable pharmacological test models, e.g. in the in vivo test model as laid down in more detail below, or in closely related test models:

Male spontaneously hypertensive rats (=SHR, insulin resistant strain from Charles River; aged 6 months) were equipped with telemetry transmitters for continuous monitoring of blood pressure and heart rate (TA11PA-C40, DSI, USA). Telemetry transmitters for continuous monitoring of blood pressure, heart rate and locomotor activity (TA11PA-C40, Data Sciences, USA) were implanted intraabdominally under inhalative halothane anesthesia. A midline abdominal incision was made, and the abdominal aorta was visualized by removal of retroperitoneal fat and connective tissue. A ligature was placed caudal of the renal arteries, the aorta was punctured with a 22G needle, and the catheter was advanced into the aorta. The entry point was sealed with tissue adhesive (Vetbond, 3M, USA), the ligature was removed, and the abdominal incision was closed. Measurements of aortic pressure were taken every 5 minutes (=min) for 4 seconds (=s) each at a sampling rate of 500 Hz, and were corrected for the corresponding ambient pressure (ambient pressure monitor, C11PR, Data Sciences, USA).

After 3 days of monitoring under baseline (untreated) conditions, animals received daglutril via their drinking water. The intended daily dose was 100 mg/kg/day of daglutril. The concentration in drinking water was adjusted once per week, resulting in an average drug intake of 98 mg/kg/day.

In a second experiment, rats were divided into two treatment groups receiving hydrochlorothiazide or hydrochlorothiazide+daglutril. Compounds were administered via the drinking water, and daily drug intake was measured by weighing the water bottles thrice weekly. Intended daily doses were 10 mg/kg/day of hydrochlorothiazide plus, in the combination group, 100 mg/kg/day of daglutril.

Concentrations of hydrochlorothiazide and daglutril in the drinking water were adjusted once per week, in order to ensure the intended daily intake of 10 and 100 mg/kg, respectively. The average daily water intake amounted to 57 and 46 ml/kg in the hydrochlorothiazide and hydrochlorothiazide+daglutril group, respectively, resulting in the uptake of 9.5 mg/kg/day of hydrochlorothiazide in the hydrochlorothiazide group, and 9.4 mg/kg/day of hydrochlorothiazide and 94.4 mg/kg/day of daglutril in the combination group.

The blood pressure, heart rate and activity values, sampled in 5 min intervals by the Dataquest™ system, were used for calculation of individual 24 hour means. These 24 hour means were exported to Excel, and group mean values of systolic blood pressure (=SBP), diastolic blood pressure (=DBP), heart rate (=HR), and locomotor activity (=ACT) were calculated for the daglutril, hydrochlorothiazide and the hydrochlorothiazide+daglutril groups. For the statistical analysis, a baseline value (pre) was calculated from the last day prior to compound application (day 3), and effects of daglutril, hydrochlorothiazide and hydrochlorothiazide+daglutril were calculated in relation to this baseline value (day 23, i.e. after 3 weeks of treatment, minus baseline value). The statistical comparison was done by using univariate ANOVA at an error level of P<0.05.

In this test model, administration of daglutril in combination with a thiazide diuretic (hydrochlorothiazide) and compared to administration of the diuretic hydrochlorothiazide only and daglutril only, showed the results as given in the following Table 1:

TABLE 1
Effects of coadministration of daglutril and a thiazide
diuretic (hydrochlorothiazide) on cardiovascular parameters
in the spontaneously hypertensive rat

| | HCTZ | | daglutril | | HCTZ + | | |
|---|---|---|---|---|---|---|---|
| CV | Only | | only | | Daglutril | | statistics |
| parameters | Mean | SEM | Mean | SEM | Mean | SEM | ANOVA |
| DBP | −7.7 | 0.6 | 2.0 | 0.5 | −9.3 ⁽*⁾ | 0.5 | P < 0.001 |
| [mmHg] | | | | | | | |
| SBP | −11.3 | 0.7 | −0.3 | 0.9 | −14.9 * | 0.7 | P < 0.001 |
| [mmHg] | | | | | | | |
| HR | −4.5 | 1.7 | −6.0 | 2.1 | −7.7 | 2.4 | n.s. |
| [1/min] | | | | | | | |

HCTZ = hydrochlorothiazide; n = 5 animals per group; SEM = Standard Error of the Mean, two-tailed ANOVA, n.s. = not significant,
⁽*⁾ P < 0.1,
* P < 0.01 two-tailed t-test HCTZ versus HCTZ + SLV306

In this test model, hydrochlorothiazide only resulted in a moderate decrease in blood pressure, daglutril only had no effect on blood pressure, while the combination group hydrochlorothiazide+daglutril showed a significantly greater decrease in blood pressure than either of the monotherapy groups. The difference in blood pressure effects between the groups was statistically significant (ANOVA, at least P<0.01).

The dosage of the active agents can depend on a variety of factors, such as mode of administration, species, age and/or individual condition. Suitable dosages for the active agents of the pharmaceutical combination according to the present invention are therapeutically effective dosages, for example those which are commercially available. Normally, in the case of oral administration, an approximate daily dose of from about 3 mg to about 2000 mg is to be estimated for each of the active agents e.g. for a patient of approximately 75 kg in weight. For example, a pharmaceutical composition according to the invention may preferably comprise daglutril as dually acting compound capable of inhibiting NEP and the endogenous endothelin producing system in the range of 50-800 mg, preferably in the range of 200-600 mg. In another preferred embodiment, a pharmaceutical composition according to the invention may preferably comprise 2-[1-(1-carboxymethyl-2-oxo-2,3,4,5-tetrahydro-1H-benzo[b]azepin-3-ylcarbamoyl)-cyclopentyl-methyl]-N-(3-dimethylamino-propyl)-N-methyl-succinamic acid as dually acting compound capable of inhibiting NEP and the endogenous endothelin producing system in the range of 30-300 mg, preferably in the range of 50-250 mg. The daily dose range of diuretics which can be used in the pharmaceutical compositions according to the invention may vary depending on i.a. the substance used and may for example be (each calculated for the pure active substance, not the salt or solvate thereof), 5-50 mg for 4-[(2-phenyl-7H-pyrrolo[2,3-d]pyrimidin-4-yl)amino]-trans-cyclohexanol; 5-50 mg for (4S)-4-hydroxy-1-(2-phenyl-7H-pyrrolo[2,3-d]pyrimidin-4-yl)-L-prolinamide; 125-2000 mg for chlorothiazide, 5-200 mg for hydrochlorothiazide, 15-200 mg for chlortalidone, 10-100 mg for xipamide, 0.5-15 mg for bumetanide, 30-1000 mg for furosemide, 2-10 mg for piretanide, 5-20 mg for torsemide, 5-20 mg for amiloride, 100-500 mg for potassium canrenoate, 100-500 mg for spironolactone, 100-1000 mg for acetazolamide or 15-200 mg for ethacrynic acid. The administration of the pharmaceutical composition may occur up to three times a day. Once daily administration forms are preferred.

### EXAMPLE I

### Capsules containing Daglutril and Hydrochlorothiazide

Capsules with the following composition per capsule are produced:

| | |
|---|---|
| Daglutril calcium salt | 250 mg |
| Hydrochlorothiazide | 50 mg |
| Corn starch | 50 mg |
| Lactose | 30 mg |
| Ethyl acetate | q.s. |

The active agents, the corn starch and the lactose are processed into a homogeneous pasty mixture using ethyl acetate. The paste is ground, and the resulting granules are placed on a suitable tray and dried at 45° C. in order to remove the solvent. The dried granules are passed through a crusher and mixed in a mixer with the further following auxiliaries:

| | |
|---|---|
| Talcum | 5 mg |
| Magnesium stearate | 5 mg |
| Corn starch | 10 mg |

and then poured into 400 mg capsules (=capsule size 0).

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A pharmaceutical composition consisting essentially of pharmacologically effective amounts of:
a) at least one neutral endopeptidase-inhibitor;
b) at least one inhibitor of the endogenous endothelin producing system, and
c) at least one diuretic.

2. A pharmaceutical composition according to claim 1, further comprising at least one pharmaceutically acceptable auxiliary or carrier.

3. A pharmaceutical composition according to claim 1, wherein said composition is suitable for oral administration, and the neutral endopeptidase-inhibitor, the inhibitor of the endogenous endothelin producing system, and the diuretic are each present in a dosage form individually selected from the group consisting of tablets, coated tablets, capsules, syrups, elixirs and suspensions.

4. A pharmaceutical composition according to claim 1, wherein the diuretic is present in a unit single dosage form physically segregated from the neutral endopeptidase-inhibitor and the inhibitor of the endogenous endothelin producing system.

5. A pharmaceutical composition according to claim 1, wherein the inhibitor of the endogenous endothelin producing system is selected from the group consisting of inhibitors of endothelin converting enzyme, inhibitors of human soluble endopeptidase and dually acting compounds capable of inhibiting endothelin converting enzyme and human soluble endopeptidase.

6. A pharmaceutical composition according to claim 1, wherein the neutral endopeptidase inhibitor (a) and the inhibitor of the endogenous endothelin producing system (b) are present in the form of a dually acting compound which inhibits both neutral endopeptidase and human soluble endopeptidase.

7. A pharmaceutical composition according to claim 6, wherein said dually acting compound which inhibits both neutral endopeptidase and the endogenous endothelin producing system is a compound corresponding to Formula I: 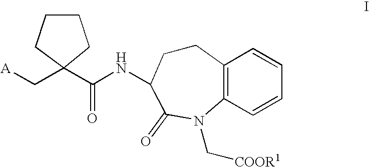
wherein
R¹ is hydrogen or a group forming a biolabile carboxylic acid ester,
A represents a group selected from the subgroups:
(a), 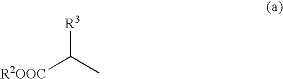
wherein
R² is hydrogen or a a group forming a biolabile carboxylic acid ester, and
R³ is a phenyl-C₁₋₄-alkyl group which can optionally be substituted in the phenyl ring by C₁₋₄-alkyl, C₁₋₄-alkoxy or halogen; or a naphthyl-C₁₋₄-alkyl group, or
(b), 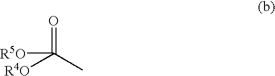
wherein
R⁴ is hydrogen or a group forming a biolabile phosphonic acid ester and
R⁵ is hydrogen or a group forming a biolabile phosphonic acid ester; or
(c), 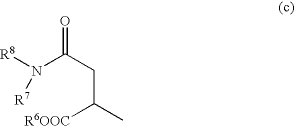
wherein
R⁶ is hydrogen or a group forming a biolabile carboxylic acid ester,
R⁷ is hydrogen, C₁₋₄-alkyl or C₁₋₄-hydroxyalkyl, the hydroxyl group of which is optionally esterified with C₂₋₄-alkanoyl or an amino acid residue, and
R⁸ is C₁₋₄-alkyl; C₁₋₄-alkoxy-C₁₋₄-alkyl; C₁₋₄-hydroxyalkyl, which is optionally substituted by a second hydroxyl group and the hydroxyl groups of which are each optionally esterified with C₂₋₄-alkanoyl or an amino acid residue; (C₀₋₄-alkyl)₂-amino-C₁₋₆-alkyl; C₃₋₇-cycloalkyl; C_{3J}-cycloalkyl-C₁₋₄-alkyl; phenyl-C₁₋₄-alkyl, the phenyl group of which is optionally substituted 1 or 2 times by C₁₋₄-alkyl, C₁₋₄-alkoxy and/or halogen; naphthyl-C₁₋₄-alkyl; C₃₋₆-oxoalkyl; phenylcarbonylmethyl, the phenyl group of which is optionally substituted 1 or 2 times by C₁₋₄-alkyl, C₁₋₄-alkoxy and/or halogen, or 2-oxoazepanyl, or
R⁷ and R⁸ together are C₄₋₇-alkylene, the methylene groups of which are optionally replaced 1 or 2 times by carbonyl, nitrogen, oxygen and/or sulfur and which are optionally substituted once by hydroxy, which is optionally esterified with C₂₋₄-alkanoyl or an amino acid residue; C₁₋₄-alkyl; C₁₋₄-hydroxyalkyl, the hydroxyl group of which is optionally esterified with C₂₋₄-alkanoyl or an amino acid residue; phenyl or benzyl,
and/or physiologically compatible salts of acids of Formula I.

8. A pharmaceutical composition according to claim 7, wherein said dually acting compound which inhibits both neutral endopeptidase and the endogenous endothelin producing system is selected from the group consisting of:
2-[1-(1-Carboxymethyl-2-oxo-2,3,4,5-tetrahydro-1H-benzo[b]azepin-3-ylcarbamoyl)-cyclopentylmethyl]-4-phenyl-butyric acid ethyl ester;
2-[1-(1-Carboxymethyl-2-oxo-2,3,4,5-tetrahydro-1H-benzo[b]azepin-3-ylcarbamoyl)-cyclopentylmethyl]-4-naphthalen-1-yl-butyric acid ethyl ester;
2-[1-(1-Carboxymethyl-2-oxo-2,3,4,5-tetrahydro-1H-benzo[b]azepin-3-ylcarbamoyl)-cyclopentylmethyl]-4-phenyl-butyric acid,
2-[1-(1-carboxymethyl-2-oxo-2,3,4,5-tetrahydro-1H-benzo[b]azepin-3-ylcarbamoyl)-cyclopentylmethyl]-4-naphthalen-1-yl-butyric acid;
and physiologically compatible salts of any of the foregoing.

9. A pharmaceutical composition according to claim 1, wherein said dually acting compound which inhibits both neutral endopeptidase and the endogenous endothelin producing system is daglutril.

10. A pharmaceutical composition according to claim 1, wherein the diuretic is selected from the group consisting of adenosine A1 antagonists, thiazides, thiazide analogues, loop diuretics, potassium sparing diuretics, carbonic anhydrase inhibitors and ethacrynic acid.

11. A pharmaceutical composition according to claim 10, wherein the diuretic is:
a) an adenosine A1 antagonist selected from the group consisting of 1,3-dipropyl-8-cyclopentylxanthine; 4-[(2-phenyl-7H-pyrrolo[2,3-d]pyrimidin-4-yl)amino]-trans-cyclo-hexanol; (4S)-4-hydroxy-1-(2-phenyl-7H-pyrrolo[2,3-d]pyrimidin-4-yl)-L-prolinamide; 8-cyclopentyl-3-N-[34(3-(4-fluorosulfonyl)benzoyl)-oxy)-propyl]-1-N-propyl-xanthine; BG-9928; CPX; FK-352; FK-453; FK-838; FR-166124; KW-3902; N-0861; WRC-0342; WRC-0571, and naxifylline; or
b) 4-[(2-phenyl-7H-pyrrolo[2,3-d]pyrimidin-4-yl)amino]-trans-cyclohexanol, or (4S)-4-hydroxy-1-(2-phenyl-7H-pyrrolo[2,3-d]pyrimidin-4-yl)-L-prolinamide; or
c) a thiazide diuretic selected from the group consisting of althiazide, bemetizide, bendroflumethiazide, benzylhydrochlorothiazide, benzthiazide, buthiazide, chlorothiazide, cyclothiazide, cyclopenthiazide, ethiazide, hydrochlorothiazide, hydroflumethiazide, methylclothiazide, paraflutizide, polythiazide, teclothiazide, and trichlormethiazide, or
d) a thiazide analogue selected from the group consisting of chloraminofenamide, chlortalidone, clofenamide, clopamide, clorexolone, fenquizone, indapamide, mefruside, metolazone, quinethazone, tripamide, and xipamide; or
e) a loop diuretic selected from the group consisting of azosemide, bumetanide, furosemide, piretanide, and torsemide; or
f) a potassium sparing diuretic selected from the group consisting of amiloride, potassium canrenoate, spironolactone, and triamterene, or
g) a carbonic anhydrase inhibitor selected from the group consisting of acetazolamide, brinzolamide, dichlorophenamide, dorzolamide, ethoxzolamide, indisulam, methazolamide, and zonisamide; or
h) a physiologically compatible tautomer or salt of any of the foregoing.

12. A pharmaceutical composition according to claim 1, wherein
the components a) and b) are present in the form of from 50 to 800 mg per day of the dually acting compound daglutirl or a physiologically compatible salt thereof which inhibits both neutral endopeptidase and the endogenous endothelin producing system, and
the diuretic c) is present in an amount of 5 to 200 mg per day in the form of hydroclorothiazide or a physiologically compatible tautomer or salt thereof.

13. A method of treating or inhibiting a cardiovascular disease, a renal disease or a liver disease selected from the group consisting of liver fibrosis and liver cirrhosis in a human or other mammal in need thereof, said method comprising administering to said human or other mammal in combination pharmacologically effective amounts of at least one neutral endopeptidase inhibitor, at least one inhibitor of the endogenous endothelin producing system, and at least one diuretic.

14. A method according to claim 13, wherein the disease is:
a) a cardiovascular disease selected from the group consisting of acute coronary syndrome; acute heart failure; angina pectoris; angina abdominalis; arrhythmias; cardiac hypertrophy; cerebral infarction; cerebral ischemias; chronic heart failure; congestive heart failure; coronary heart disease; critical leg ischemia; hypertension selected from the group consisting of essential hypertension, pulmonary hypertension, renal hypertension and hypertension associated with obesity, insulin resistance or diabetes; myocardial infarction; restenosis and stroke; or
b) a renal disease selected from the group consisting of diabetic nephropathy; acute renal failure; chronic renal failure; ischemic renal failure; and renal diseases due to exposure to a toxic substance.

15. A method according to claim 13, wherein the neutral endopeptidase inhibitor, the inhibitor of the endogenous endothelin producing system, and the diuretic are administered simultaneously.

16. A method according to claim 13, wherein the neutral endopeptidase inhibitor, the inhibitor of the endogenous endothelin producing system, and the diuretic are administered separately in succession.

17. A method according to claim 13, wherein the neutral endopeptidase inhibitor and the inhibitor of the endogenous endothelin producing system are administered in the form of a dually acting compound which inhibits neutral endopeptidase and human soluble endopeptidase.

18. A method according to claim 13, wherein a fixed combination is administered comprising:
daglutril and hydrochlorothiazide; or
daglutril and 4-[(2-phenyl-7H-pyrrolo[2,3-d]pyrimidin-4-yl)amino]-trans-cyclohexanol; or
daglutril and (4S)-4-hydroxy-1-(2-phenyl-7H-pyrrolo[2,3-d]pyrimidin-4-yl)-L-prolinamide; or
2-[1-(1-carboxymethyl-2-oxo-2,3,4,5-tetrahydro-1H-benzo[b]azepin-3-ylcarbamoyl)-cyclopentylmethyl]-N-(3-dimethylamino-propyl)-N-methyl-succinamic acid and hydrochlorothiazide; or
2-[1-(1-carboxymethyl-2-oxo-2,3,4,5-tetrahydro-1H-benzo[b]azepin-3-ylcarbamoyl)-cyclopentylmethyl]-N-(3-dimethylamino-propyl)-N-methyl-succinamic acid and 4-[(2-phenyl-7H-pyrrolo[2,3-d]pyrimidin-4-yl)amino]-trans-cyclohexanol; or
2-[1-(1-carboxymethyl-2-oxo-2,3,4,5-tetrahydro-1H-benzo[b]azepin-3-ylcarbamoyl)-cyclopentylmethyl]-N-(3-dimethylamino-propyl)-N-methyl-succinamic and (4S)-4-hydroxy-1-(2-phenyl-7H-pyrrolo[2,3-d]pyrimidin-4-yl)-L-prolinamide; or
any of their physiologically compatible tautomers or salts.

19. A kit comprising in separate containers in a single package pharmaceutical dosage forms for use in combination, comprising:
i1) a first separate container containing a pharmaceutical dosage form comprising at least one neutral endopeptidase inhibitor, and a second separate container containing a pharmaceutical dosage form comprising at least one inhibitor of the endogenous endothelin producing system,
or
i2) a first separate separate container containing a pharmaceutical dosage form comprising a dually acting compound which inhibits both neutral endopeptidase and the endogenous endothelin producing system;
and
ii) a further separate container containing a pharmaceutical dosage form comprising at least one diuretic.

20. A kit according to claim 19, further comprising a leaflet indicating that the at least one neutral endopeptidase inhibitor and the at least one inhibitor of the endogenous endothelin producing system or the dually acting compound capable of inhibiting neutral endopeptidase and the endogenous endothelin producing system may be administered in combination with the at least one diuretic.
